(19) United States
(12) Patent Application Publication
(10) Pub. No.: US 2023/0245752 A1
(43) Pub. Date: Aug. 3, 2023

(54) SYSTEM AND METHOD FOR BIOMARKER ANALYSIS IN SPORTING, WELLNESS AND HEALTHCARE

(71) Applicant: Ai-Genetika Inc., doing business as BioTwin, Québec (CA)

(72) Inventors: Louis-Philippe Noel, Québec (CA); Manon Fradin, Québec (CA); Camille Houle, Québec (CA)

(73) Assignee: Ai-Genetika Inc., doing business as BioTwin, Québec, QC (CA)

(21) Appl. No.: 18/162,026

(22) Filed: Jan. 31, 2023

Related U.S. Application Data
(60) Provisional application No. 63/267,343, filed on Jan. 31, 2022.

(51) Int. Cl. G16H 20/30; G01N 33/68; G16H 50/20; G16H 50/30

(52) U.S. Cl. CPC G16H 20/30; G01N 33/6893; G16H 50/20; G16H 50/30

(57) ABSTRACT

Artificial intelligence systems and computer-implemented methods for generating digital twins, establishing wellness parameters and generating recommendations to improve performance and wellness. An artificial intelligence system comprises a biosample collection unit, a physical analysis unit to perform a detection, relative quantification and untargeted analysis of analytes from the biosamples, a computing unit comprising: an automatic data collection unit, a data analysis unit for analysing the data, and a real-time data computing integration unit which automatically integrates the data generated from the analysis, transforms the data to establish representing values of wellness parameters through time, compares the representing values with data stored in the automatic data collection unit and responds to a change by generating a set of recommended actions or patterns response to improve performance and wellness. Full or partial phenotyped digital twins can be compared to knowledge bases to assess an organism over time or to assess another organism.

### CROSS REFERENCE TO RELATED APPLICATION

This application claims the benefit of U.S. Provisional application 63/267,343, filed on Jan. 31, 2022.

### FIELD OF THE INVENTION

The invention relates to a system and method for rapidly detecting, identifying, quantifying and analyzing analytes and/or biomarkers from dry blood spots (DBS), blood, urine, saliva, tears, sweat, and other type of biosample from a living organism for the generation of a digital twin. This invention more particularly relates to the system and method for collecting, processing and interpreting data from biosamples analysis, and the use of the acquired data for the generation of digital twins of a living organism such an animal or a human being, wherein use of artificial intelligence (AI) provides an on-going monitoring, analysis, prediction and personalized recommendations for sports training, wellness, consumer health, nutritional supplement companies and healthcare.

### BACKGROUND OF THE INVENTION

Detection and identification of biomolecules from the microbiome and metabolites from samples is widely used for diagnosis and monitoring of diseases.

The use of “multi-omics” profiling technologies (genomics, proteomics, metabolomics, epigenomic etc.) has exploded in recent years and begun to advance understanding of disease and reactions to lifestyles, nutrition, genetics, exercise and environment.

Concurrently, artificial intelligence is known to be used to simulate outcomes and to compare data sets with known data sets in order to draw conclusions based on previous knowledge and ongoing knowledge acquisition (machine learning acquisition). For example, in the healthcare sector, detection of high levels of cholesterol in blood can be a predictor of the risk of eventual atherosclerosis and heart disease. Artificial intelligence would predict that abnormally rising blood cholesterol levels are a predictor of heart disease. Furthermore, genetics, diets and exercise levels are also known to modulate blood cholesterol. Such correlations are known and studied for a number of diseases or conditions. As such, it is known to use artificial intelligence to provide risk assessment and recommendations based on specific test data collected on patients.

The expression “digital twin” refers to digital data representation of a physical object, machine or living organisms such as animals or human beings.

A “digital twin” in the context of a living organism such as a human being is a collection of digital information indicative of health or other health-related parameters like age, fitness, body mass index, history of disease, genetics, etc. It is a digital model based on real-world measurements (known as artifacts) that provides a dynamic representation over time of the physiological status of a subject. Each human being can be provided with a digital twin comprising a modest or large amount of data on various biomarkers such as levels of various enzymes, hormones, minerals, etc. There are about 115,000 known human metabolites that have been identified and over 1.5M that are estimated and non identified. Biomarkers can be proteins, fats, nutrients, waste products, hormones, gene variants, and various levels of detectable species or metabolites present in the body, in secretions or excretions.

Integration of digital twins in healthcare has also been discussed by Croatti et al. in Journal of Medical Systems 44,161 (2020) in an article entitled On the Integration of Agents and Digital Twins in Healthcare. The article discussed the context of strategic planning by creating a digital twin of a hospital and running simulations on the digital replica to provide more effective care interventions. Meanwhile, Bjornsson, B., et al, discussed the use of genotyping to provide personalized medicine in Digital twins to personalize medicine, Genome Medicine 12, 4 (2020).

In Deep Digital Phenotyping and Digital Twins for Precision Health: Time to Dig Deeper, (J Med Internet Res 2020; 22(3):e16770) Fagherazzi, emphasizes the growing importance of digital twin for providing precision medicine and personalized therapeutic strategy. The article stresses the known desideratum of personalized medicine, but does not provide a complete method and system as presented herein.

However, so far, the use of digital twins and artificial intelligence has been limited. For example, a device commercialized under the brand Lumen™ is a small Bluetooth-connected device similar in shape to a vape pen where users exhale. The device measures CO₂ levels. The CO₂ level indicates if the user's metabolism being in fat burning mode or carbohydrate burning mode. A fat-burning mode is said to be directly related to a lower level of CO₂, and also to weight loss. The device also comes with a specific smartphone application which provides diet recommendations and scores based on the CO₂ reading. This is directed towards athletes or the weight-loss industry. Of note, the apparatus and method only focus on one parameter, namely CO₂, and as such does not create a digital twin.

Another known service is provided by “Lets get Checked”™. It provides the kind of home testing that is traditionally provided by health care establishments. Thus, it is a home testing kit mailed to clients who have selected one or more tests to be performed. Fluid samples (such as blood or urine) are directly collected by the user/client. After self-collection, samples are mailed back to a lab for analysis, process and medical check. Up to 50 biomarkers can be measured in total within various tests. The biomarkers include hormones, cholesterol, bacteria counts, etc. The company reports to clients their results with emphasis on abnormal results and findings of infection. They also recommend repeated testing over time to track important biomarkers variations such as cholesterol levels. However, the reports provide readings of individual biomarkers without providing profiles of combined biomarkers.

On the genetic side, companies such as “23andMe”™ provide home kits mailed to clients who collect saliva samples and mail back the kit for genetic testing. A laboratory analyzes saliva sample to determine the genotype and variant identification and reports every genetic anomalies that may be statistically linked to certain diseases. Because gene expression and diseases can also be modulated by environmental factors and lifestyle, the results are not fully predictive of outcome and doesn't reflect the phenotype. Indeed, genetic sequencing does not take into account other biomarkers, especially the ones that may change over time nor profiles of multiple biomarkers.

The “MolecularYou”™ service provides detailed reports based on blood tests. These reports use various biomarkers present in blood and rely on a combination of genotyping and traditional biomarker measurements. Clients receive a report which summarizes a high level snapshot of disease risks and recommendations. This report includes, an organ health assessment, risk of contracting specific diseases, inflammation score, medications that may pose a risk, and individual test results compared with optimal ranges.

The iCarbonX™ service is collecting a large amount of biomarker data such as DNA, RNA, proteins, microbiomes, etc. and is using artificial intelligence to develop correlations between theses biomarkers and specific disease state. ICarbonX then issues recommendations based on these findings.

The Onegevity™ service provides biometric data measurements, and use artificial intelligence to describe the state of an individual's health and issuance of personalized wellness recommendations. The service allows for home sample collection of microbiomes, blood and saliva which are mail back to a lab for results and recommendations to improve health and wellness.

Other services of blood contents measurement analysis include for example: Nightingale™ and Thriva™ combine blood analysis technology, identify disease risks and make comparisons with ideal ranges of blood components.

One skilled in the art would appreciate that the above described services do not feature a holistic phenotype profiling of a digital twin or a living organism, so as to provide more accurate predictions overtime and issue recommendations on the health and wellness of an individual on a given time. The combination of data obtained from multiple untargeted multi-omics and multiple metabolomes measured from a living organism over a period of time to create a complete digital twin representation provides a full phenotype of a subject, which may be used to predict any changes and issue recommendations on a specific time frame.

Thus, despite recent advancements and desired progress in this area, there remains a need for novel and improved methods for generating a personalized and more complete digital twin to effectively monitor, analyse, assess, predict parameters and outcomes that are useful in sports training, wellness or healthcare settings and precision medicine.

There is also a need for developing improved artificial intelligence directed to the same purposes.

Also, there is a need for supporting professionals involved in sports training or injury rehabilitation, wellness and medicine by providing ready access to biomarker data and artificial intelligence to predict and improve patient or client outcomes, or assess physiological cycles of a living organism in order to improve performance.

The present invention addresses these needs and other needs as it will be apparent from the review of the disclosure and description of the features of the invention hereinafter.

### BRIEF SUMMARY

In a general sense, the present technology provides an artificial intelligence system for establishing wellness parameters of a living organism and generating recommendations to improve performance and wellness of said living organism, the system comprising:
a sample collection unit for collecting biosamples (either dry and/or liquid) from said living organism;
at least one analysis unit adapted to read and analyze the content of the sample collection unit, wherein the at least one analysis unit performs a detection, relative quantification and untargeted analysis of analytes from the biosamples;
a computing unit coupled to the sample collection unit and/or the at least one analysis unit, the computing unit comprising:

an automatic data collection unit to collect and store data generated from the detection, relative quantification and untargeted analysis of analytes from the biosamples;

a data analysis unit comprising multiple detection means for analysing the data generated from the detection, relative quantification and untargeted analysis of analytes from the biosamples; and
a real-time data computing integration unit coupled to the computing unit and in electronic communication with the automatic data collection unit and/or the data analysis unit, wherein the real-time data computing integration unit provides for

automatically integrating the data generated from the detection, relative quantification and untargeted analysis of analytes from the biosamples;

transforming data generated to establish representing values of wellness parameters of said living organism through time;

comparing the representing values with data stored in the automatic data collection unit to determine any changes; and

responding to a change of the representing values of wellness parameters of the living organism by generating a set of recommended actions or patterns response to improve performance and wellness of said living organism.

Also provided is a computer-implemented method for establishing wellness parameters of a living organism and generating recommendations to improve performance and wellness of said living organism, the method comprising:
acquiring at least a first set of data generated during a first detection, relative quantification and untargeted analysis of analytes from the biosamples of said living organism;
transforming the at least first set of data to establish a first set of representing values of wellness parameters of said living organism through time;
receiving a second set of data generated during a second detection, relative quantification and untargeted analysis of analytes from the biosamples of said living organism the computer system;
transforming the second set of data to establish a second representing values of wellness parameters of said living organism;
comparing the first and second representing values to determine any changes; and
responding to a change between the first and second representing values of wellness parameters of the living organism by generating a set of recommended actions or patterns response to improve performance and wellness of said living organism.

Also provided is a computer-implemented method for creating at least one predicted digital twin phenotype of a living organism, comprising, acquiring at least one set of data generated during a detection, relative quantification and untargeted analysis of analytes from the biosamples of said living organism, transforming the set of data to establish at least one set of representing values of wellness parameters of said living organism, providing a comparative knowledge base of analytes and performance, wellness and health parameters over a large number of subjects, retrieving, from the comparative knowledge base, prediction data for at least a portion of the large number of subjects, comprising knowledge base analytes data and knowledge base values of wellness and health parameters for the said portion of the large number of subjects over at least two distinct points in time per subject, generating at least one predicted digital twin phenotype, comprising determining, from the prediction data, at least one trend of the knowledge base analytes data or the knowledge base values of wellness and health parameters, modifying the values of wellness parameters of the living organism according to the at least one trend according to a predetermined time step and composing a predicted digital twin comprising modified values of wellness parameters so obtained.

In some embodiments, the artificial intelligence, the system further comprises a comparative knowledge base of analytes and performance, wellness and health parameters over a large number of subjects or a comparative knowledge base on predictors, diseases or conditions. In some embodiments, the comparative knowledge base further comprises records of actions or patterns responses for a large number of subjects.

In some embodiments, the system is configured to generate one or more predicted digital twin phenotypes corresponding to one or more future points in time based on at least one of the first or second values of wellness parameters.

In some embodiments, the method further comprises providing at least one predetermined digital twin or at least one predetermined set of representing values of wellness parameters of the living organism, generating two or more trends or two or more of said predicted digital twins, performing statistical analysis on the two or more trends or on the two or more predicted digital twins and determining a probability of at least one action or pattern response resulting in the predetermined digital twin or the at least one predetermined set of representing values of wellness parameters of the living organism.

Additional aspects, advantages and features of the present invention will become more apparent upon reading of the following non-restrictive description of preferred embodiments which are exemplary and should not be interpreted as limiting the scope of the invention.

### BRIEF DESCRIPTION OF THE FIGURES

In order for the invention to be readily understood, embodiments of the invention are illustrated by way of example in the accompanying figures.

FIG. 1 is a flowchart of the artificial intelligence system in accordance to one particular embodiment.

FIG. 2 is an illustration of the health and wellness parameters that can be established using a digital twin.

FIG. 3 is an illustration of the sources of biomarkers and their counterpart measurements.

FIG. 4 is an illustration of the importance of metabolites in a digital twin phenotyping.

Further details of the invention and its advantages will be apparent from the detailed description included below.

### DETAILED DESCRIPTION OF EMBODIMENTS

In the following description of the embodiments, references to the accompanying figures are illustrations of one or more examples by which the invention may be practiced. It will be understood that other embodiments may be made without departing from the scope of the invention disclosed. Unless defined otherwise, all technical and scientific terms used herein have the same meaning as commonly understood by one of ordinary skill in the art to which the invention belongs.

In a most general sense, the invention provides an artificial intelligence system and a computer-implemented method to effectively detect and predict physiological conditions, outcomes and variations in sports training, wellness and healthcare of a living organism. The invention also provides means for the detection, relative quantification and untargeted analysis of analytes from the biosamples provided by or taken from a living organism, and in some instances issuing recommendation for preventing the appearance, or for facilitating the disappearance, proliferation or manifestations of diseases conditions in living organisms.

When used herein, the term “biomarker” is meant to refer to useful measurements from tissue or fluid biosamples linked to a specific phenotype. It includes excretions or secretions from a live being, including, without being exhaustive, levels of minerals, hormones, proteins, fats, sugars, vitamins, metabolites, microbiomes, analytes, enzymes, antigens and antibodies and including various molecular markers and cells. Biomarkers can also include gene variants, alleles and other genetic information. Biomarkers can also include species such as telomeres and their length measurements.

When used herein, the term “analyte” is meant to refer to the chemical substances or molecules that are being analyzed in a sample. They can be a single compound or a mixture of compounds, and they can be found in various biological fluids, such as blood, urine, and saliva, from a living organism. The purpose of analyzing analytes is to obtain information about the presence and concentration of specific substances.

When used herein, the term “wellness” is meant to be general and refer for example to overall health, sleep, nutrition, life expectancy, biological age, energy levels, body mass index or other similar measurements, flexibility, strength and other similar features.

The term “sport” is meant to be general and refer to competitive and non-competitive sports or other physical activities.

The term “health” is meant to be general and refer to all parameters and biomarkers indicative of health or disease states or progression thereof.

In the context of the present specification, a “server” is a computer program that is running on appropriate hardware and is capable of receiving requests (e.g., from electronic devices) over a network (e.g., a communication network), and carrying out those requests, or causing those requests to be carried out. The hardware may be one computer or one computer system, but neither is required to be the case with respect to the present technology. In the present context, the use of the expression a “server” is not intended to mean that every task (e.g., received instructions or requests) or any particular task will have been received, carried out, or caused to be carried out, by the same server (i.e., the same software and/or hardware); it is intended to mean that any number of software elements or hardware devices may be involved in receiving/sending, carrying out or causing to be carried out any task or request, or the consequences of any task or request; and all of this software and hardware may be one server or multiple servers, both of which are included within the expressions “at least one server” and “a server”.

In the context of the present specification, “electronic device” is any computing apparatus or computer hardware that is capable of running software appropriate to the relevant task at hand. Thus, some (non-limiting) examples of electronic devices include general purpose personal computers (desktops, laptops, netbooks, etc.), mobile computing devices, smartphones, and tablets, and network equipment such as routers, switches, and gateways. It should be noted that an electronic device in the present context is not precluded from acting as a server to other electronic devices. The use of the expression “an electronic device” does not preclude multiple electronic devices being used in receiving/sending, carrying out or causing to be carried out any task or request, or the consequences of any task or request, or steps of any method described herein. In the context of the present specification, a “client device” refers to any of a range of end-user client electronic devices, associated with a user, such as personal computers, tablets, smartphones, and the like.

In the context of the present specification, the expression “computer readable storage medium” (also referred to as “storage medium” and “storage”) is intended to include non-transitory media of any nature and kind whatsoever, including without limitation RAM, ROM, disks (CD-ROMs, DVDs, floppy disks, hard drivers, etc.), USB keys, solid state-drives, tape drives, etc. A plurality of components may be combined to form the computer information storage media, including two or more media components of a same type and/or two or more media components of different types.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, the expression “information” includes information of any nature or kind whatsoever capable of being stored in a database. Thus, information includes, but is not limited to audiovisual works (images, movies, sound records, presentations etc.), data (location data, numerical data, etc.), text (opinions, comments, questions, messages, etc.), documents, spreadsheets, lists of words, etc.

In the context of the present specification, the expression “communication network” is intended to include a telecommunications network such as a computer network, the Internet, a telephone network, a Telex network, a TCP/IP data network (e.g., a WAN network, a LAN network, etc.), and the like. The term “communication network” includes a wired network or direct-wired connection, and wireless media such as acoustic, radio frequency (RF), infrared and other wireless media, as well as combinations of any of the above.

In the context of the present specification, the term “application” refers to a software present on an electronic device and provided with a user interface. The application can rely on processing means to process data and issue information. The application may also communicate over a communication network with a database.

In the context of the present specification, the words “first”, “second”, “third”, etc. have been used as adjectives only for the purpose of allowing for distinction between the nouns that they modify from one another, and not for the purpose of describing any particular relationship between those nouns. Thus, for example, it should be understood that, the use of the terms “server” and “third server” is not intended to imply any particular order, type, chronology, hierarchy or ranking (for example) of/between the server, nor is their use (by itself) intended imply that any “second server” must necessarily exist in any given situation. Further, as is discussed herein in other contexts, reference to a “first” element and a “second” element does not preclude the two elements from being the same actual real-world element. Thus, for example, in some instances, a “first” server and a “second” server may be the same software and/or hardware, in other cases they may be different software and/or hardware.

In the context of the present specification, the word “about” when used in relation to numerical designations or ranges means the exact numbers plus or minus experimental measurement errors and plus or minus 10 percent of the exact numbers.

In the context of the present specification, the term “untargeted analysis” provides for the detection, quantification and multi-omics analysis of a plurality of compounds, thereby providing a detailed picture of the health and wellness of an individual, including, but not limited to genomic predispositions and a reading of the metabolome which is a representation of what is actually happening in the body at a given time.

In the context of the present specification, the term “targeted analysis” provides for a metabolomic analysis of specific compounds, while ignoring other compounds present in the sample analysed.

In the context of the present specification, “relative quantification and analysis” of the biosamples from said living organism comprises characterizing the sample (qualitatively and quantitatively) by performing a plurality of measurements such as measuring the pH of the sample, photo or scan profiling by spectroscopy, performing a metabolomics and/or proteomics analysis by liquid-chromatography coupled with mass spectrometry (LC-MS) and/or ion mobility spectrometry-Mass Spectrometry (IMS-MS), and/or Nuclear Magnetic Resonance (NMR), performing a single nucleotide polymorphism (SNP) microarray analysis with evaluation of risk score (PRS), single cell ARN sequencing, or a combination thereof.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams or illustrations represent conceptual views of the principles of the present technology. Similarly, it will be appreciated that any diagrams, flowcharts, and the like represent various processes which may be substantially represented in computer-readable media and so executed by a computer or processor, whether or not such computer or processor is explicitly shown.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

The present invention is based on the holistic collection of a number of useful analytes which are analyzed and depicted in a form of an individual profile otherwise known as a digital twin profile. This digital twin can be used to generate a phenotype profiling of a living organism. This refers to providing as many analytes readings and analysis as possible to generate a phenotype digital twin.

The number of analytes that are featured in a digital twin profile can vary, for example from about 10 to about 100 to about 1000 or more depending on the level of detail required. A fully phenotyped digital twin may not be necessary before drawing useful information from the present invention.

The digital twin profile can be rebuilt at various time intervals to track variations over time. These variations can result for example from disease progression, lifestyle or training changes, aging, nutrition modifications, weight variations, physical and hormonal cycles, exercise and/or medications.

Instead of focusing on individual analytes and making predictions or recommendations based on the measurement(s), the present invention is directed to the establishment and tracking of a digital twin profile of a holistic combination of analytes. Thus, in one embodiment, the present invention provides a system for measuring and creating a digital twin profile providing a phenotyped digital twin.

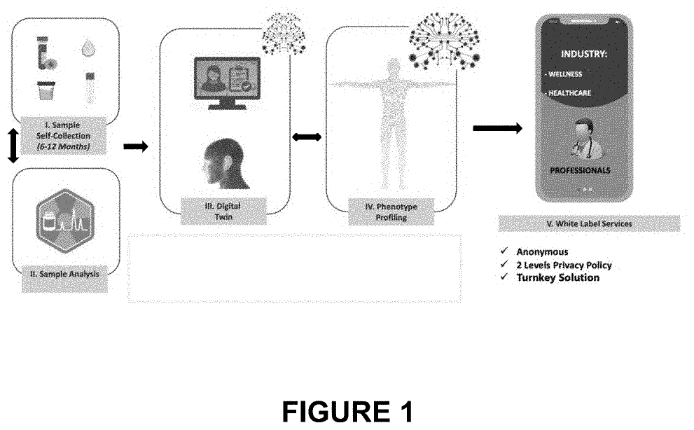

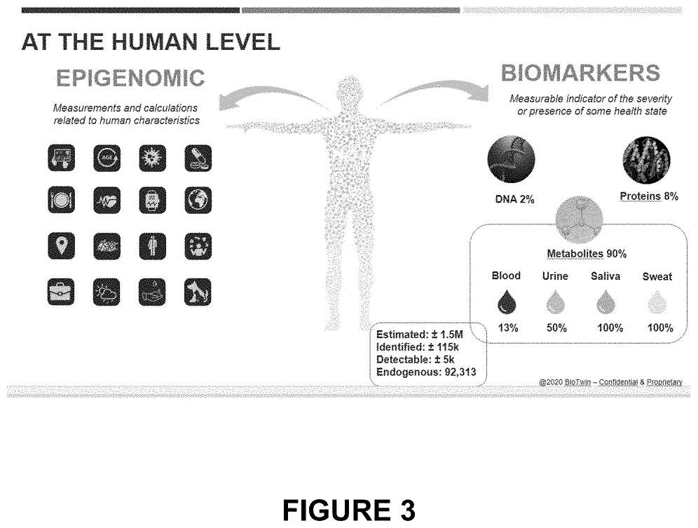

Referring to FIG. 1, in embodiments of the present invention, the service is provided for sports training, therapy, rehabilitation, wellness or medical professionals. Clients or patients receive a sampling/testing kit for self-collection of a sample at any location. The sampling/testing kit can be provided by a professional or ordered from a third party and delivered to for sampling. As illustrated in FIG. 3, many biofluid samples can be collected, such as blood, saliva, tears, sweat, vaginal cultures, semen or urine. Tissue samples can include for example hair, follicles, nails, shed skin or tissues obtained by biopsy. Microbiome samples can for example be stool samples. Sample collection is performed at any location by the living organism itself, and the analysis may be performed with the assistance of a computing system or a computing application. Alternatively, sample collection may occur at a designated location or facility where professional assistance is provided.

In practice, the sampling/testing kit may be coupled to an artificial intelligence computing unit for performing the analysis of the sample, which may be performed with the assistance of the a computing system or a computing application at any location Alternatively, samples may be returned by mail to a laboratory for testing and analysing the analytes at a facility. Apart from comparing the analytes readings to preferred and predetermined ranges, all analytes are added to a consortium of data and results that forms a digital twin of the individual living organism.

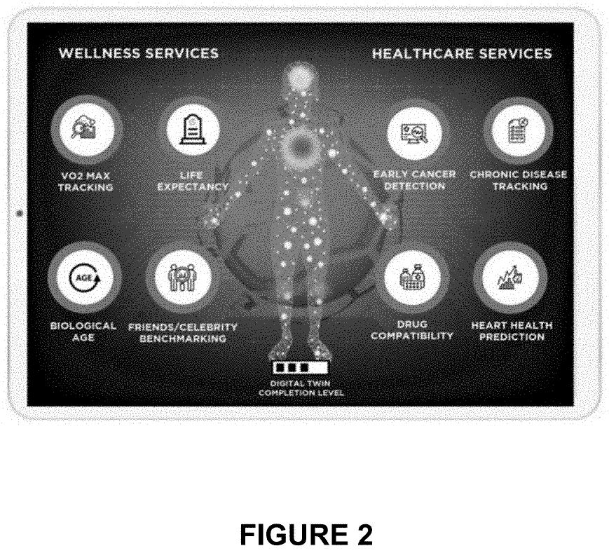

In preferred embodiments, a sufficiently large number of analytes are measured and recorded to provide a suitable basis for accurate phenotyping of the digital twin. Referring to FIG. 2, incomplete phenotyping can be illustrated by a bar graph or otherwise to signal to the living organism or professional assisting the living organism that the phenotyping is yet to be completed.

In preferred embodiments, the phenotyping is provided by graphical representation such as bar graph plotting all analytes (not shown). This graph, in some embodiments a bar graph or histogram, provides a unique phenotyping of the individual living organism, akin to a fingerprint.

This technique can be repeated over time at some intervals such as, for example, every day, once every week, every three or six months so as to provide a shifting of the shape of the phenotyping in response to individual recommendations, such as nutrition, diet, sleep, exercise, training, rest, and other lifestyle or medication recommended by the laboratory or a health professional that follows the living organism.

Referring again to FIG. 1, in preferred embodiments, artificial intelligence, including machine learning are used to compare known outcomes and predictions to individual phenotyping essentially by comparing the graphical shape of the analyte phenotyping or by statistical analysis. Comparing the graphical shape of the analyte phenotyping is akin to fingerprint comparisons with software to find matches and correlations. The modifications over time of the analyte phenotyping can also be used to predict outcomes and predispositions towards specific or general health parameters or conversely towards disease apparition or progression. These modifications can also be used to run simulations based on trend analysis and artificial intelligence where a database and machine learning algorithms (MLAs) are used for predictions and simulations.

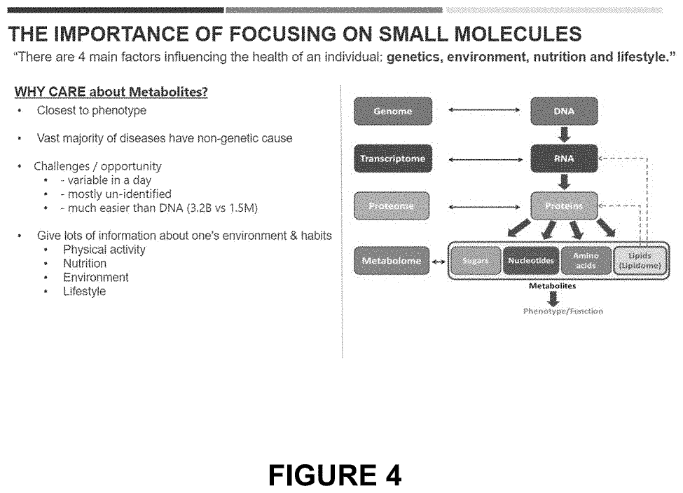

In embodiments, as illustrated in FIG. 4, the previously described system can be used by sports trainers, therapists, rehabilitation or wellness professionals to predict and improve various factors such as whole health index, biological age and life expectancy measurements, body composition analysis, energy balance, performance, libido, drive, stress management, sleep quality and quantity overtraining, aerobic capacity and benchmarking of these in comparison to known celebrities, friends, training partners or others.

In embodiments, the above described system can be used by medical professionals to predict and improve various factors such as disease risks and factors such as heart health, heart disease, cardiovascular disease, stroke, hemorrhage, mental disease, cancer, chronic diseases or conditions such as chronic pain, fibromyalgia, autoimmune diseases such as lupus, asthma, eczema, psoriasis, Crohn's, colitis, diverticulitis, constipation, diarrhea, allergies, intolerances, Parkinson disease, Alzheimer, dementia, drug treatment compatibility, dietary needs, exercise or lifestyle requirements, injury, inflammation, infection, virus load, antibodies and vaccine requirements.

In practice, the system of the present invention empowers sports trainers, therapists, rehabilitation, wellness and medical experts to provide more informed and personalized precision interventions for their clients and patients. These experts can include for example, sports therapists, physiotherapists, chiropractors, osteopaths and related professionals. By tracking the biomarker phenotyping over time, professionals can consider and appraise the effect of recommendations, treatments and simulations of treatments by being proactive rather than reactive.

In one or more embodiments of the phenotyping system, collected analyte data is entered in a network having access to a processor having access to a set of machine learning algorithms (MLAs) having been trained to determine the optimal health and wellness parameters at the time of processing or over time and based on the age and other parameters of the living organism.

In one or more embodiments, the set of machine learning algorithms (MLAs) has been trained to provide predictions and simulations of health and analyte outcomes over time. The set of MLAs is also used to provide recommendations to various aspects of the living organism nutrition, exercise, rest, sleep, relaxation, medication or fluid intake so as to provide improved outcomes in terms of analytes and other sports performance such as VO₂ Max, wellness and health parameters.

In embodiments, the system of the present invention provides a database and MLAs for storing analytes data and a comparative knowledge base of analytes and performance, wellness and health parameters over a large number of subjects and a comparative knowledge base on predictors and diseases or conditions. The database is constantly improved upon by the MLAs so as to become more accurate.

In embodiments, the ensemble of analyte data collected for a specific living organism provides a digital twin. This digital twin is compared to the knowledge bases to provide an assessment, over time, of the living organism, or it may also be used as benchmark for assessing another living organism in a given time. The assessment is done by graphical comparison or statistical data analysis of the digital twin in comparison with known phenotypes and diseases or conditions. One or more processor is used in conjunction with the database to provide data outputs and results and to run simulations or outcome predictions such as probable disease progression. The processor also provides recommended changes to various controllable parameters such as nutrition, treatments, medications, sleep, rest, fluid intake, training, exercise and types, duration, intensity levels of training and exercise. These recommended changes are designed to improve performance, health and wellness outcomes, over time, for the living organism, or another living organism.

The database is accessible via a network and via electronic communication means.

In embodiments, the system of the present invention also provides a software product such as a smartphone application providing data, results, predictions, simulations and guidance. In a preferred embodiment, the application provides a living organism module with content and functionalities for the living organism and a second related application for the professional or a third party.

Referring again to FIG. 2, the living organisms version of the application provides functionalities, data and results on features of the digital twin and recommended dietary needs, exercise or lifestyle requirements, injury treatment, and vaccine or other requirements. The application also provides tracking of data over time and comparison with preferred data values or comparisons with known celebrities, friends, training partners or others, as benchmarks. The application also provides basic simulation functions showing how data changes can affect the overall digital twin and lead to improved outcomes for the living organism.

The professional version of the application provides extended functionalities, prediction and simulation functions and recommended treatments or regimens or general advice to increase performance. For example, the extended simulation functions allows the professional to canvass various scenarios and better understand the effect and leverage of various analytes on real world outcomes. These functionalities allow the professional to provide precise interventions with the living organism.

In embodiments, there is provided an artificial intelligence system for establishing wellness parameters of a living organism and generating recommendations to improve performance and wellness of said living organism, the system comprising:
a sample collection unit for collecting biosamples (either dry and/or liquid) from said living organism;
at least one analysis unit adapted to read and analyze the contents of the sample collection unit, wherein the at least one analysis unit performs a detection, relative quantification and untargeted analysis of analytes from the biosamples;
a computing unit coupled to the sample collection unit and/or the at least one analysis unit, the computing unit comprising:

an automatic data collection unit to collect and store data generated from the detection, relative quantification and untargeted analysis of analytes from the biosamples;

a data analysis unit comprising multiple detection means for analysing the data generated from the detection, relative quantification and untargeted analysis of analytes from the biosamples; and
a real-time data computing integration unit coupled to the computing unit and in electronic communication with the automatic data collection unit and/or the data analysis unit, wherein the real-time data computing integration unit provides an automatic intergradation of the data generated from the detection, relative quantification and untargeted analysis of analytes from the biosamples, a transformation of the data generated to establish representing values of wellness parameters of said living organism through time, comparing the representing values with data stored in the automatic data collection unit to determine any changes, and responding to a change of the representing values of wellness parameters of the living organism by generating a set of recommended actions or patterns response to improve performance and wellness of said living organism.

The living organism may be a human subject or an animal.

The untargeted analysis of the collected biosamples from said living organism may comprise detecting a plurality of analytes from the living organism after collecting at least one sample from the living organism.

The untargeted analysis of the biosamples from said living organism may comprise measuring the pH of the sample, photo or scan profiling by spectroscopy, performing a metabolomics and/or proteomics analysis by liquid-chromatography coupled with mass spectrometry (LC-MS) and/or ion mobility spectrometry-Mass Spectrometry (IMS-MS) and/or Nuclear Magnetic Resonance (NMR), performing a single nucleotide polymorphism (SNP) microarray analysis with evaluation of risk score (PRS), single cell ARN sequencing, or a combination thereof.

The analyte may be a biomarker contained and obtained from dry blood spots (DBS), blood, urine, saliva, tears, sweat, or another type of biosample from a living organism.

The data generated from the detection, relative quantification and untargeted analysis of analytes from the biosamples from said living organism may compose a digital twin phenotype of said living organism.

The data generated from the detection, relative quantification and untargeted analysis of analytes from the biosamples from said living organism may be indicative of parameters useful to benchmark sports performance or sports therapy or other forms of therapy, of wellness parameters, or health or disease results or parameters or progression thereof.

The time to establish the wellness parameters of said living organism may be a one-time sampling event or a plurality of sampling events.

The set of recommended actions or patterns response to improve performance and wellness of said living organism may be useful to benchmark the performance and wellness of another living organism.

The set of recommended actions or patterns response to improve performance and wellness of said living organism may be useful to predict the performance and wellness of another living organism.

The set of recommended actions or patterns response to improve performance and wellness of said living organism may improve sporting, therapy, wellness and healthcare outcomes of said living organism or another living organism.

The another living organism may be human subject or an animal.

The use of the system may provide an on-going monitoring, analysis, prediction and personalized recommendations for sports training, wellness, consumer health, nutritional supplement companies and healthcare.

In another embodiment, there is provided a computer-implemented method for establishing wellness parameters of a living organism and generating recommendations to improve performance and wellness of said living organism, the method comprising:
acquiring at least a first set of data generated during a first detection, relative quantification and untargeted analysis of analytes from the biosamples of said living organism;
transforming the at least first set of data to establish a first set of representing values of wellness parameters of said living organism through time;
receiving a second set of data generated during a second detection, relative quantification and untargeted analysis of analytes from the biosamples of said living organism the computer system;
transforming the second set of data to establish a second representing values of wellness parameters of said living organism;
comparing the first and second representing values to determine any changes; and
responding to a change between the first and second representing values of wellness parameters of the living organism by generating a set of recommended actions or patterns response to improve performance and wellness of said living organism.

The living organism may be a human subject or an animal.

The at least one untargeted analysis of the collected biosamples from said living organism may comprise obtaining and detecting a plurality of analytes from the living organism after collecting at least one biosample from the living organism.

The second untargeted analysis of the biosamples from said living organism may comprise obtaining and detecting a plurality of analytes from the living organism after collecting at least one biosample from the living organism.

The untargeted analysis of the biosamples from said living organism may comprise measuring the pH of the sample, photo or scan profiling by spectroscopy, performing a metabolomics and/or proteomics analysis by liquid-chromatography coupled with mass spectrometry (LC-MS) and/or ion mobility spectrometry-Mass Spectrometry (IMS-MS), and/or Nuclear Magnetic Resonance (NMR), performing a single nucleotide polymorphism (SNP) microarray analysis with evaluation of risk score (PRS), single cell ARN sequencing, or a combination thereof.

The liquid, dry or gas analytes may be biomarkers contained in and obtained from dry blood spots (DBS), blood, urine, saliva, tears, sweat, and other types of biosample from a living organism.

The data generated from the at least one and/or from the second detection, relative quantification and untargeted analysis of analytes from the biosamples from said living organism may compose a digital twin phenotype of said living organism.

The data generated from the at least one and/or from the second detection, relative quantification and untargeted analysis of analytes from the biosamples from said living organism may be indicative of parameters useful to benchmark sports performance or sports therapy or other forms of therapy, of wellness parameters, or health or disease results or parameters or progression thereof.

The time to establish the wellness parameters of said living organism may be a one-time sampling event or a plurality of sampling events.

The set of recommended actions or patterns response to improve performance and wellness of said living organism may improve sporting, therapy, wellness and healthcare outcomes of said living organism or another living organism.

The set of recommended actions or patterns response to improve performance and wellness of said living organism may be useful to benchmark the performance and wellness of another living organism.

The set of recommended actions or patterns response to improve performance and wellness of said living organism may be useful to predict the performance and wellness of another living organism.

The another living organism may be a human subject or an animal.

The use of the computer-implemented method may provide for an on-going monitoring, analysis, prediction and personalized recommendations for sports training, wellness, consumer health, nutritional supplement companies and healthcare.

In another embodiment, there is provided a sampling/testing kit unit for use with the artificial intelligence system as disclosed herein, the sampling/testing kit unit is couplable to a computing unit for collecting and performing an analysis of a liquid, dry and/or gas analytes from the living organism.

In another embodiment, there is provided a sampling/testing kit unit for use with the computer-implemented method as disclosed herein, the sampling/testing kit unit couplable to a computing unit for collecting and performing a physical analysis of a liquid, dry and/or gas analytes from the living organism.

Those skilled in the art will recognize, or be able to ascertain, using no more than routine experimentation, numerous equivalents to the specific procedures, embodiments, claims, and examples described herein. Such equivalents are considered to be within the scope of this invention and covered by the claims appended hereto.

1. An artificial intelligence system for establishing wellness parameters of a living organism and generating recommendations to improve performance and wellness of said living organism, the system comprising:
a sample collection unit for collecting liquid biosamples, dry biosamples, or a combination thereof from said living organism;
at least one analysis unit adapted to receive and analyze the contents of the sample collection unit, wherein the at least one analysis unit performs a detection, relative quantification and untargeted analysis of analytes from the biosamples;
a computing unit coupled to the sample collection unit and/or the at least one analysis unit, the computing unit comprising:
an automatic data collection unit to collect and store data generated from the detection, relative quantification and untargeted analysis of analytes from the biosamples;
a data analysis unit comprising multiple detection means for analysing the data generated from the detection, relative quantification and untargeted analysis of analytes from the biosamples; and
a real-time data computing integration unit coupled to the computing unit and in electronic communication with the automatic data collection unit and/or the data analysis unit, wherein the real-time data computing integration unit provides
for automatically integrating the data generated from the detection, relative quantification and untargeted analysis of analytes from the biosamples,
for transforming data generated to establish representing values of wellness parameters of said living organism through time,
for comparing the representing values with data stored in the automatic data collection unit to determine any changes, and
for responding to a change of the representing values of wellness parameters of the living organism by generating a set of recommended actions or patterns response to improve performance and wellness of said living organism.

2. The artificial intelligence system of claim 1, wherein the living organism is a human subject or an animal.

3. The artificial intelligence system of claim 2, wherein the untargeted analysis of the collected biosamples from said living organism comprises obtaining and analyzing a plurality of analytes obtained from the living organism by collecting at least one biosample from the living organism.

4. The artificial intelligence system of any one of claim 3, wherein the untargeted analysis of the biosamples from said living organism comprises measuring the pH of the sample, photo or scan profiling by spectroscopy, performing a metabolomics and/or proteomics analysis by liquid-chromatography coupled with mass spectrometry (LC-MS) and/or ion mobility spectrometry-Mass Spectrometry (IMS-MS) and/or Nuclear Magnetic Resonance (NMR), performing a single nucleotide polymorphism (SNP) microarray analysis with evaluation of risk score (PRS), single cell ARN sequencing, or a combination thereof.

5. The artificial intelligence system of claim 4, wherein the analytes are selected from the group consisting of biomarkers contained in and obtained from dry blood spots (DBS), blood, urine, saliva, tears and/or sweat.

6. The artificial intelligence system of claim 5, wherein the system composes a digital twin phenotype of said living organism from the data generated from the detection, relative quantification and untargeted analysis of analytes from the biosamples from said living organism.

7. The artificial intelligence system of claim 6, wherein the time to establish the wellness parameters of said living organism is a one time sampling event or a plurality of sampling events.

8. The artificial intelligence system of claim 7, wherein use of the system provides an on-going monitoring, analysis, prediction and personalized recommendations for sports training, wellness, consumer health, nutritional supplement companies and healthcare.

9. A computer-implemented method for establishing wellness parameters of a living organism and generating recommendations to improve performance and wellness of said living organism, the method comprising the steps of:
acquiring at least a first set of data generated during a first detection, relative quantification and untargeted analysis of analytes obtained from biosamples of said living organism;
transforming the at least first set of data to establish a first set of representing values of wellness parameters of said living organism through time;
receiving a second set of data generated during a second detection, relative quantification and untargeted analysis of analytes from the biosamples of said living organism the computer system;
transforming the second set of data to establish second representing values of wellness parameters of said living organism;
comparing the first and second representing values to determine any changes; and
responding to a change between the first and second representing values of wellness parameters of the living organism by generating a set of recommended actions or patterns response to improve performance and wellness of said living organism.

10. The computer-implemented method of claim 9, wherein the living organism is a human subject or an animal.

11. The computer-implemented method of claim 10, wherein the at least one untargeted analysis of the collected biosamples from said living organism comprises obtaining and analyzing a plurality of analytes obtained from the living organism by collecting at least one biosample from the living organism.

12. The computer-implemented method of claim 11, wherein the second untargeted analysis of the collected biosamples from said living organism comprises obtaining and analyzing a plurality of analytes obtained from the living organism by collecting at least one biosample from the living organism.

13. The computer-implemented method of claim 12, wherein the untargeted analysis of the biosamples from said living organism comprises measuring the pH of the sample, photo or scan profiling by spectroscopy, performing a metabolomics and/or proteomics analysis by liquid-chromatography coupled with mass spectrometry (LC-MS) and/or ion mobility spectrometry-Mass Spectrometry (IMS-MS), and/or Nuclear Magnetic Resonance (NMR), performing a single nucleotide polymorphism (SNP) microarray analysis with evaluation of risk score (PRS), single cell ARN sequencing, or a combination thereof.

14. The computer-implemented method of claim 13, wherein the analytes are selected from the group consisting of biomarkers contained in and obtained from biosamples from dry blood spots (DBS), blood, urine, saliva, tears and/or sweat.

15. The computer-implemented method of claim 14, wherein the data generated from the at least one and/or from the second detection, relative quantification and untargeted analysis of analytes from the biosamples from said living organism establishes a digital twin phenotype of said living organism.

16. The computer-implemented method of claim 15, wherein the data generated from the at least one and/or from the second detection, relative quantification and untargeted analysis of analytes from the biosamples from said living organism is indicative of parameters useful to benchmark sports performance or sports therapy or other forms of therapy, of wellness parameters, or health or disease results or parameters or progression thereof.

17. A biosampling kit unit for use with the artificial intelligence system claim 1, the biosampling kit unit being connectable to the at least one analysis unit, the biosampling kit further containing instructions for use of the biosampling kit.

18. A computer-implemented method for creating at least one predicted digital twin phenotype of a living organism, the method comprising:
(a) acquiring at least one set of data generated during a detection, relative quantification and untargeted analysis of analytes from biosamples of said living organism;
(b) transforming the at least one set of data to establish at least one set of representing values of wellness parameters of said living organism;
(c) providing a comparative knowledge base of analytes and performance, wellness and health parameters over a large number of subjects;
(d) retrieving, from the comparative knowledge base, prediction data for at least a portion of the large number of subjects comprising knowledge base analytes data and knowledge base values of wellness and health parameters for the said portion of the large number of subjects over at least two distinct points in time per subject;
(e) generating at least one predicted digital twin phenotype, the generating comprising:
(i) determining, from the prediction data, at least one trend of said knowledge base analytes data or said knowledge base values of wellness and health parameters;
(ii) modifying said values of wellness parameters of said living organism according to said at least one trend according to a predetermined time step;
(iii) composing a predicted digital twin comprising modified values of wellness parameters obtained in step (ii).

19. The method according to claim 18, wherein the knowledge base further comprises records of actions or patterns responses for said large number of subjects.

20. The method according to claim 19, further comprising:
(a) providing at least one predetermined digital twin or at least one predetermined set of representing values of wellness parameters of said living organism;
(b) generating two or more of said trends or two or more of said predicted digital twins;
(c) performing statistical analysis on said two or more trends or on said two or more predicted digital twins;
(d) determining a probability of at least one of said actions or patterns responses resulting in the predetermined digital twin or the at least one predetermined set of representing values of wellness parameters of said living organism.
